(19) United States
(12) Patent Application Publication
(10) Pub. No.: US 2002/0032177 A1
(43) Pub. Date: Mar. 14, 2002

(54) Use of D-chiro-inositol in the treatment of conditions associated with hypothalamic gene expression

(75) Inventors: Geoffrey Allan, Richmond, VA (US); Charles V. Mobbs, New York, NY (US)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: Insmed Pharmaceuticals, Inc.

(21) Appl. No.: 09/844,292

(22) Filed: Apr. 30, 2001

Related U.S. Application Data
(60) Provisional application No. 60/200,471, filed on Apr. 28, 2000.

(51) Int. Cl. A61K 31/715; A61K 31/66; A61K 31/357; A61K 31/28; A61K 31/215; A61K 31/075; A61K 31/047

(52) U.S. Cl. 514/129; 514/102; 514/54; 514/464; 514/492; 514/548; 514/715; 514/729

(57) ABSTRACT

The present invention relates to the use of D-chiro-inositol (DCI) as a regulator of hypothalamic gene expression and, more particularly, to the use of DCI and derivatives and metabolites thereof to treat neuroendocrine impairments such as obesity, polycystic ovarian syndrome (PCOS), impaired regulation of hormones during aging and to correct such neuroendocrine impairments and associated functions.

### CROSS-REFERENCE TO RELATED APPLICATIONS

This application claims benefit under 35 U.S.C. § 119(e) of U.S. Provisional Application No. 60/200,471, filed Apr. 28, 2000.

### BACKGROUND OF THE INVENTION

1. Field of the Invention

The present invention relates to the use of D-chiro-inositol (DCI) as a regulator of hypothalamic gene expression and, more particularly, to the use of DCI and derivatives and metabolites thereof to treat neuroendocrine impairments such as obesity, polycystic ovarian syndrome (PCOS), impaired regulation of hormones during aging and to correct such neuroendocrine impairments and associated functions.

2. Background Art

Obesity, especially upper body obesity, is a common and very serious public health problem in the United States and throughout the world. According to recent statistics, more than 25% of the United States population and 27% of the Canadian population are overweight (Kuczmarski Amer. J. Clin. Nutr. 55:495S502S (1992); Reeder et. al., Can. Med. Ass. J. 23: 226-233 (1992)). Upper body obesity is the strongest risk factor known for type II diabetes mellitus, and is a strong risk factor for cardiovascular disease and cancer as well. Recent estimates for the medical cost of obesity are $150,000,000,000 world wide. The problem has become serious enough that the surgeon general has begun an initiative to combat the ever increasing obesity rampant in American society.

Much of this obesity induced pathology can be attributed to the strong association with dyslipidemia, hypertension, and insulin resistance. Many studies have demonstrated that reduction in obesity by diet and exercise reduces these risk factors dramatically. Unfortunately, these treatments are largely unsuccessful with a failure rate reaching 95%. This failure may be due to the fact that the condition is strongly associated with genetically inherited factors that contribute to increased appetite, preference for highly caloric foods, reduced physical activity, and increased lipogenic metabolism. This indicates that people inheriting these genetic traits are prone to becoming obese regardless of their efforts to combat the condition. Therefore, a pharmacological agent that can correct this adiposity handicap and allow the physician to successfully treat obese patients in spite of their genetic inheritance is needed.

Insulin plays a major role in the regulation of satiety through its effects on hypothalamic gene expression. Insulin-deficient individuals tend to consume more calories and exhibit a profile of hypothalamic gene expression typical of fasting which is reversed by direct infusion of insulin into the hypothalamus. The hypothalamus controls not only food intake but also other mechanisms including hormone secretion, mobilization of the sympathetic nervous system, regulation of basal metabolic rate—all of which functions are dependent on insulin action (i.e., they are all altered in diabetic and fasting individuals).

Insulin resistance is a well known risk factor for endocrine impairments such as obesity and polycystic ovarian syndrome (PCOS). Metformin, which increases sensitivity to insulin, decreases food intake in obese rodents and humans and partially restores fertility in women with PCOS. These observations suggest that an insulin sensitizer which preferentially enhances the effects of insulin on the hypothalamus may form the basis of a pharmaceutical treatment for neuroendocrine impairments such as obesity, PCOS, the impaired regulation of hormones during aging, and the correction of neuroendocrine impairments and associated functions. Ideally, such drugs would increase the sensitivity of the hypothalamus to insulin without increasing the sensitivity of adipose tissue.

Both fasting and genetic obesity are associated with the following neuroendocrine profile: elevated NPY and AGRP mRNA and decreased POMC mRNA in hypothalamus; and elevated glucocorticoid and decreased sex, thyroid, and growth hormones in blood. This profile acts as an anabolic force, which tends to preserve metabolic resources in a fasted individual but in fed individuals causes obesity. This profile develops in fasting because insulin is low in fasting and insulin acts to counter the fasting/obesity profile. There is therefore a need for treatment which enhances insulin sensitivity and reverse the obesity-causing anabolic profile.

Leptin is the product of the obese (ob) gene and is secreted by adipose cells (Zhang et al. Nature 372:425-432 (1994)). Leptin receptors are found in the choroid plexus and the hypothalamus. (Tartaglia, L. A. Cell 83:1263-1271 (1995)). The action of leptin to regulate energy balance appears to be primarily through effects in the brain, in particular the hypothalamus. A rising level of leptin, as triglyceride stores increase, is proposed to serve as a negative feedback signal to the brain, resulting in decreased food intake, increased energy expenditure and resistance to obesity. In addition, circulating leptin appears to play an important role in the neuroendocrine axis (Ahima, R. S., et al., Nature 382:250-252 (1996)). It has also been shown that in humans, serum leptin concentrations vary with the percentage of body fat, and that during weight loss, serum leptin concentrations initially decline, but increase again during maintenance of the lower weight (Considine et al. N. Eng. J. Med. 334:292-295 (1996)). Increased leptin levels can be achieved by the administration of exogenous leptin or, alternatively, by increasing endogenous leptin production, for example by stimulating the endogenous gene to produce increased amount of leptin.

Mice which are homozygous for the ob gene (ob/ob) are obese, perhaps due to an underexpression of leptin. When ob/ob mice are given daily injections of recombinant protein, their food intake was markedly inhibited and they experienced a reduction in body weight and fat. In lean (i.e. wild-type) mice, daily injections of leptin lead to modest decreases of food intake and body weight. The results for body fat have been contradictory. (Pelleymounter et al., Science 269:540-543 (1995); Halaas et al., Science 269:543-546 (1995); and Campfield et al., Science 269:546-549 (1995).

Dieting is often an ineffective way to control obesity in the long term, since the loss of weight causes the hypothalamus to respond as if the body were fasting, pushing the body to eat more and regain the lost weight. There is therefore a need for blocking the hypothalamic response to weight loss and preventing dieting individuals from regaining lost weight.

### BRIEF SUMMARY OF THE INVENTION

It has now been found that certain isomers of inositol, namely D-chiro-inositol (DCI) and derivatives and metabolites thereof and compounds containing DCI or a derivative or metabolite thereof, have significant effects on mammalian endocrinology and metabolism. More specifically, it has now been found that the administration of DCI to subjects suffering from certain metabolic diseases or disorders results in improved regulation of hypothalamic gene expression.

Accordingly, a first embodiment of the present invention is directed to a method of improving hypothalamic gene expression in a mammal, particularly a human, by administering to that mammal an effective amount of D-chiro-inositol (DCI), or a derivative or metabolite thereof.

A second embodiment of the present invention is directed to a method of controlling the weight of a mammal, particularly a human, by administering to that mammal an effective amount of D-chiro-inositol (DCI), or a derivative or metabolite thereof.

A third embodiment of the present invention is directed to a method of increasing serum leptin in a mammal, particularly a human, by administering to that mammal an effective amount of D-chiro-inositol (DCI), or a derivative or metabolite thereof.

A fourth embodiment of the present invention is directed to a method for the prophylaxis or treatment of diseases or disorders associated with abnormal hypothalamic gene expression, including obesity, PCOS, the impaired regulation of hormones during aging and the correction of neuroendocrine impairments and associated functions, by administering to a mammal in need thereof an effective amount of D-chiro-inositol (DCI), or a derivative or metabolite thereof.

A fifth embodiment of the present invention is directed to a pharmaceutical composition comprising an effective amount of D-chiro-inositol (DCI), or a derivative or metabolite thereof.

### BRIEF DESCRIPTION OF THE FIGURES

FIG. 1 is a graph showing the effect of DCI on lowering levels of NPY mRNA in ob/ob mice.

FIG. 2 is a graph showing the effect of DCI on lowering levels of AGRP mRNA in ob/ob mice.

FIG. 3 is a graph showing the effect of DCI on decreasing levels of leptin mRNA in ob/ob mice.

FIG. 4 is a graph showing the effect of DCI on increasing levels of plasma leptin in ob/ob mice.

### DETAILED DESCRIPTION OF THE INVENTION

In a first preferred embodiment, the present invention is directed to a method of improving hypothalamic gene expression in a mammal, particularly a human, comprising the step of administering to that mammal an effective amount of D-chiro-inositol (DCI), or a derivative or metabolite thereof.

In a second preferred embodiment, the present invention is directed to a method of controlling the weight of a mammal, particularly a human, comprising the step of administering to that mammal an effective amount of D-chiro-inositol (DCI), or a derivative or metabolite thereof.

In a third preferred embodiment, the present invention is directed to a method of increasing serum leptin in a mammal, particularly a human, comprising the step of administering to that mammal an effective amount of D-chiro-inositol (DCI), or a derivative or metabolite thereof.

In a fourth preferred embodiment, the present invention is directed to a method for the prophylaxis or treatment of diseases or disorders associated with abnormal hypothalamic gene expression, including obesity, PCOS, the impaired regulation of hormones during aging and the correction of neuroendocrine impairments and associated functions, comprising the step of administering to a mammal in need thereof an effective amount of D-chiro-inositol (DCI), or a derivative or metabolite thereof.

Preferably, each of the inventive methods comprises the step of administering to a mammal in need thereof an effective amount of DCI. While the inventive methods preferably involves administration of DCI per se, however, suitable derivatives and/or metabolites of DCI, including compounds containing DCI, may also be employed as desired.

As used herein, a “suitable derivative or metabolite” of D-chiro-inositol is a compound based on or derived from the D-chiro-inositol moiety that provides the desired physiological effect. Illustrative examples of suitable derivatives and metabolites of D-chiro-inositol include, but are not limited to, the following: D-chiro-inositol phosphates; D-chiro-inositol esters, preferably acetates; D-chiro-inositol ethers, preferably lower alkyl ethers; D-chiro-inositol acetals; D-chiro-inositol ketals, and compounds containing D-chiro-inositol. Suitable derivatives and metabolites of DCI for use in the inventive methods may be determined empirically by those skilled in the art.

As used herein, a “compound containing D-chiro-inositol” is any compound that contains the D-chiro-inositol moiety. Illustrative examples of D-chiro-inositol containing compounds include, but are not limited to, the following: polysaccharides containing D-chiro-inositol and one or more additional sugars, such as glucose, galactose and mannose, or derivatives thereof, such as glucosamine, galactosamine and mannitol; D-chiro-inositol phospholipids; and complexes or chelates of D-chiro-inositol with one or more metal ions and the like.

The active agent employed in the inventive methods (i.e. D-chiro-inositol or a suitable derivative or metabolite thereof) may be used alone or in admixture with one or more additional active agents. For example, according to the methods of the present invention, D-chiro-inositol may be administered in combination with an insulin sensitizer, such as metformin.

The term “effective amount” as used herein means that amount of DCI that elicits the biological or medicinal response in a tissue, system, animal or human that is being sought by a researcher, veterinarian, medical doctor or other clinician, which includes alleviation of the symptoms of the disease or condition being treated. In general, DCI is preferably administered at a daily dosage of from about 0.001 to about 20 mg/kg of body weight, preferably given in a single dose or in divided doses two to six times a day, or in sustained release form; in the case of an adult human, the total daily dose will generally be from about 0.07 milligrams to about 3500 milligrams of DCI, preferably from about 0.1 mg to about 1000 mg, and most preferably from about 10 mg to about 500 mg.

As used herein, the term “composition” is intended to encompass a product comprising the specified ingredients in the specified amounts, as well as any product which results, directly or indirectly, from combination of the specified ingredients in the specified amounts.

The pharmaceutical compositions employed in the methods of the present invention also preferably include a pharmaceutically acceptable excipient, such as those disclosed in the Handbook of Pharmaceutical Excipients, American Pharmaceutical Association, 2nd Ed. (1994), which is herein incorporated by reference in its entirety. The pharmaceutical composition preferably takes the form of solid tablets and/or capsules suitable for oral administration, although liquid formulations are also possible. The composition may be prepared into a form suitable for oral administration by any conventional method known to the art.

These pharmaceutical compositions will be formulated and dosed in a fashion consistent with good medical practice, taking into account the clinical condition of the individual patient (especially the side effects of treatment with the active agent), the site of delivery of the composition, the method of administration, the scheduling of administration, and other factors known to practitioners. The “effective amount” of each active agent (i.e. DCI or a derivative thereof) for the purposes of the present invention is determined in view of such considerations. Those skilled in the art can readily determine empirically an appropriate “effective amount” of each active agent for a particular mammalian patient.

DCI for use according to the present invention may also be administered pharmacologically as a prodrug. The expression “prodrug” as used herein denotes a derivative of DCI which is converted to DCI in vivo by an enzymatic or chemical process but exhibits enhanced delivery characteristics and/or therapeutic value. The preparation and administration of prodrugs of saccharides, for example in the form of methylated or acetylated hydroxyl groups, is well known in the art. (Baker, D. C. et al. (1984) J. Med. Chem. 27 270-274).

As used herein, the phrase “pharmaceutically acceptable” is intended to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, a “pharmaceutically acceptable carrier” is a pharmaceutically acceptable material, composition or vehicle, such as a liquid or solid filler, diluent, excipient, solvent or encapsulating material, involved in carrying or transporting the active agents of the inventive compositions from one organ, or portion of the body, to another organ, or portion of the body. Each carrier must be “acceptable” in the sense of being compatible with the other ingredients of the formulation and not injurious to the patient.

Some illustrative examples of materials which can serve as pharmaceutically-acceptable carriers include, but are not limited to, the following: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the inventive pharmaceutical compositions.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, and the particular mode of administration. The amount of active ingredients which can be combined with a carrier material to produce a single dosage form will generally be that amount of each active ingredient which, together, produce the desired therapeutic effect. Generally, out of one hundred per cent, this amount will range from about 0.01 per cent to about ninety-nine percent of active ingredients, preferably from about 0.1 per cent to about 90 per cent, most preferably from about 1 per cent to about 90 per cent.

In general, the formulations are prepared by uniformly and intimately bringing into association the active ingredient with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of each active ingredient. The active ingredients of the inventive compositions may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as those disclosed in the Handbook of Pharmaceutical Excipients, American Pharmaceutical Association, 2nd Ed. (1994), and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents.

In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredients can also be in micro-encapsulated form, if appropriate, with one or more of the above-described excipients.

Liquid dosage forms for oral administration of the inventive compositions include pharmaceutically acceptable emulsions, microemulsions, solutions, suspensions, syrups and elixirs. In addition to the active ingredients, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active ingredients, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal or vaginal administration may be presented as a suppository, which may be prepared by mixing the active ingredients of the present invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active ingredients. Formulations which are suitable for vaginal administration also include pessaries, tampons, creams, gels, pastes, foams or spray formulations containing such carriers as are known in the art to be appropriate.

Dosage forms for the topical or transdermal administration of DCI include powders, sprays, ointments, pastes, creams, lotions, gels, solutions, patches and inhalants. The active ingredient may be mixed under sterile conditions with a pharmaceutically-acceptable carrier, and with any preservatives, buffers, or propellants which may be required.

Ophthalmic formulations, eye ointments, powders, solutions, drops, sprays and the like, are also contemplated as being within the scope of this invention.

Pharmaceutical compositions suitable for parenteral administration comprise DCI (or a derivative thereof) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Illustrative examples of suitable aqueous and nonaqueous carriers which may be employed in the pharmaceutical compositions of the invention include, but are not limited to, the following: water; ethanol; polyols, such as glycerol, propylene glycol, polyethylene glycol, and the like, and suitable mixtures thereof, vegetable oils, such as olive oil; and injectable organic esters, such as ethyl oleate. Proper fluidity can be maintained, for example, by the use of coating materials, such as lecithin, by the maintenance of the required particle size in the case of dispersions, and by the use of surfactant.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

When DCI, or a derivative or metabolite thereof, is administered as a pharmaceutical, to humans and animals, it can be given alone or as a pharmaceutical composition containing, for example, 0.01 to 99.5% (more preferably, 0.1 to 90%) of each active ingredient together in combination with at least one pharmaceutically acceptable carrier.

The preparations of the present invention may be given orally, parenterally, topically, or rectally. They are of course given by forms suitable for each administration route. Oral administration is particularly preferred.

The phrases “parenteral administration” and “administered parenterally” as used herein are intended to mean modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein are intended to mean the administration of a compound, drug or other material other than directly into the central nervous system, such that it enters the patient's system and, thus, is subject to metabolism and other like processes, for example, subcutaneous administration.

The pharmaceutical compositions may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the active ingredients of the present invention, which may be used in a suitable hydrated form, and/or the pharmaceutical compositions used in the methods of the present invention, are formulated into pharmaceutically acceptable dosage forms by conventional methods known to those of skill in the art.

As noted, actual dosage levels of the active ingredient in the pharmaceutical compositions may be varied so as to obtain an amount which is effective to achieve the desired therapeutic response for a particular patient, composition, and mode of administration, without being toxic to the patient.

The selected dosage level will depend upon a variety of factors, including, but not limited to, the following: the activity of DCI (or the derivative thereof);

the route of administration; the time of administration; the rates of absorption, distribution, metabolism and/or excretion of the particular active ingredient being employed; the duration of the treatment; other drugs, compounds and/or materials used in combination with the particular active ingredients employed; the age, sex, weight, condition, general health and prior medical history of the patient being treated; and like factors well known in the medical arts.

A physician or veterinarian having ordinary skill in the art can readily determine the effective amount of the active ingredient required in the pharmaceutical compositions. For example, the physician or veterinarian could start doses of the pharmaceutical composition at levels lower than that required in order to achieve the desired therapeutic effect and gradually increase the dosage until the desired effect is achieved.

If desired, the effective daily dose of the active ingredients may be administered as two, three, four, five, six or more sub-doses administered separately at appropriate intervals throughout the day, optionally, in unit dosage forms.

Therapeutic compositions can be administered with medical devices known in the art. For example, a therapeutic composition of the present invention can be administered with a needleless hypodermic injection device, such as the devices disclosed in U.S. Pat. Nos. 5,399,163; 5,383,851; 5,312,335; 5,064,413; 4,941,880; 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4.,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variableflow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are well known to those skilled in the art.

The DCI pharmaceutical compositions of the present invention may also include at least one other pharmacologically-active agent, such as dieting agents, hormone replacement agents, insulin, leptin, metformin, and the like.

### EXAMPLES

### Animal Models

Animal-based model systems of body weight disorders may include, but are not limited to, non-recombinant and engineered transgenic animals.

Non-recombinant animal models for body weight disorders may include, for example, genetic models. Such genetic body disorder models may include, for example, mouse models of obesity such as mice homozygous for the autosomal recessive ob, db, or tub alleles.

Non-recombinant, non-genetic animal models of body weight disorders may include, for example, rat models in which bilateral lesions exist in the ventromedial hypothalamus, leading to hyperphagia and gross obesity, or in which ventrolateral hypothalamus lesions exist, which lead to aphagia. Further, mice which, as newborns, are fed mono-sodium-glutamate (MSG) develop obesity, and may, therefore, also be utilized as animal models for body weight disorders.

Additionally, animal models exhibiting body weight disorder-like symptoms may be engineered by utilizing, for example, target gene sequences in conjunction with techniques for producing transgenic animals that are well known to those of skill in the art. For example, target gene sequences may be introduced into, and overexpressed in, the genome of the animal of interest, or, if endogenous target gene sequences are present, they may, either be overexpressed or, alternatively, may be disrupted in order to underexpress or inactivate target gene expression.

The ob/ob mouse is a model of obesity and diabetes that is known to carry an autosomal recessive trait linked to a mutation in the sixth chromosome.

Recently, Yiying Zhang and co-workers published the positional cloning of the mouse gene linked with this condition (Zhang, Y., et al., Nature 372:425-32 (1994)). Physiologists have postulated for years that, when a mammal overeats, the resulting excess fat signals to the brain that the body is obese which, in turn, causes the body to eat less and burn more fuel (Hervey, G. R., Nature 227:629-631 (1969)). This “feedback” model is supported by parabiotic experiments, which implicate a circulating hormone controlling adiposity.

A high calorie diet (compositionally equivalent to the typical American diet) will induce obesity and diabetes in some mouse strains (e.g., C57BL/6J) but not others (e.g., A/J). The diet-susceptible strains develop insulin resistance, whereas the diet-resistant strains do not. In diet-resistant (insulin-sensitive) mice, the diet induces a hypothalamic gene profile which resist obesity (i.e., decreased NPY and elevated POMC), whereas in diet susceptible (insulin-resistant) mice, this compensatory change in hypothalamic gene expression does not occur. These changes in hypothalamic gene expression are ultimately regulated by insulin, although part of the effect may be mediated indirectly by the effect of insulin on leptin synthesis in fat, which then feeds back to hypothalamus to enhance the direct effect of insulin on the hypothalamus. By enhancing the rate of translation and secretion of leptin this hypothalamic response is blocked or attenuated.

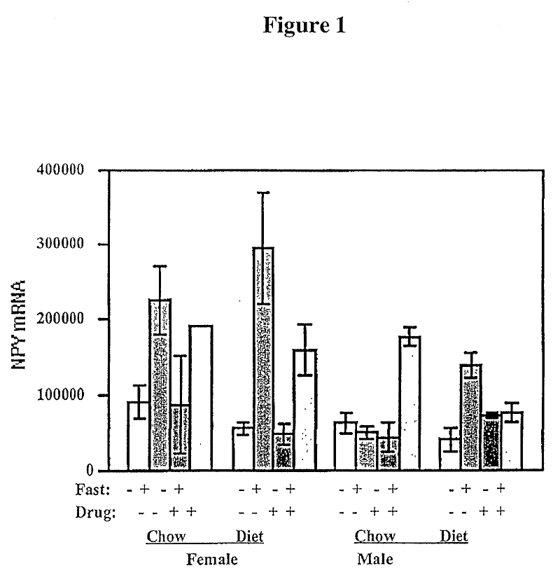

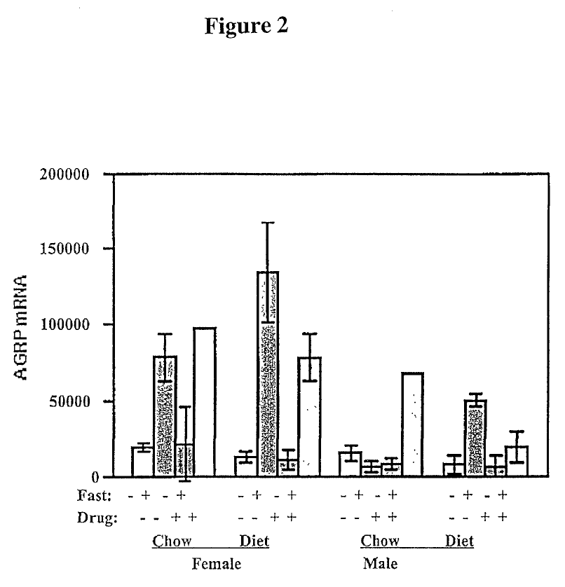

FIG. 1 and FIG. 2 depicts the results of studies in the diet induced mice on NPY and AGRP mRNA levels. DCI leads to lower NPY and AGRP mRNA. As shown in the Figures, these effects are most apparent in the fasted state, where DCI wither blocks or attenuates the effect of fasting in the diet-induced obese mice.

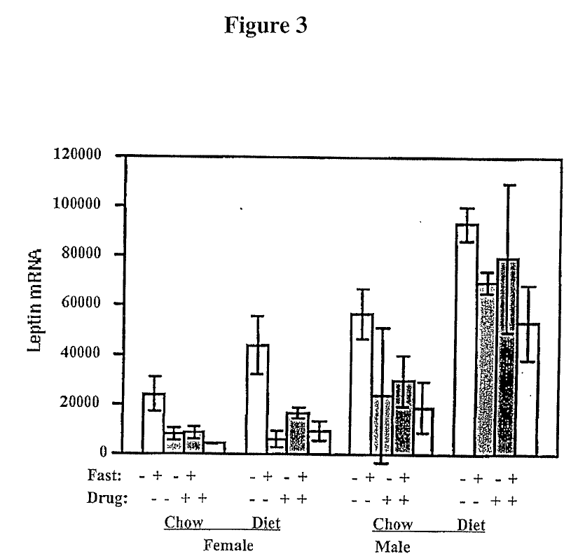

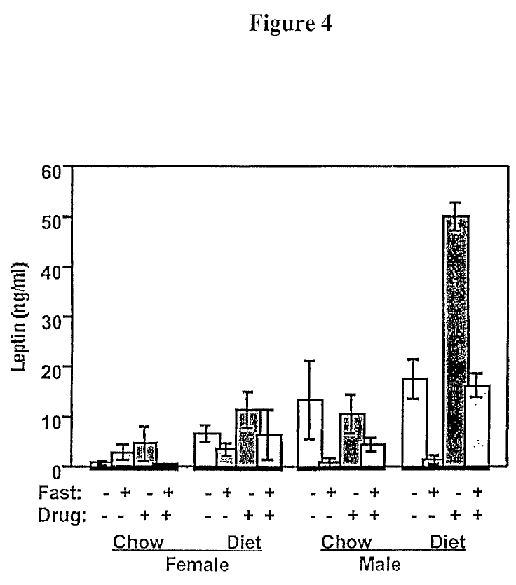

FIG. 3 and FIG. 4 depict, respectively, the effect of DCI administration on leptin mRNA and plasma leptin in mice. DCI increased plasma leptin over 2-fold in fed, and over 5-fold in 48-hour fasted mice without increasing adiposity. Surprisingly, DCI actually caused a decrease in leptin mRNA while increasing plasma leptin. This result is understood since experimental elevation of leptin by injection is known to produce pronounced decreases in leptin mRNA, at least partially independent of adiposity. Thus, DCI enhances the rate of translation and secretion of leptin.

During aging, adiposity decreases dramatically, leading to decreased leptin and associated hypothalamic and neuroendocrine changes. There is an association in humans between falling leptin and the age-associated increase in cortisol and decrease in T3. Therefore, DCI administration in those cases will lead to correction of neuroendocrine impairments and associated functions. Younger people with low plasma leptin (and associated neuroendocrine impairments, including infertility and in some cases, like the Pima Indians, possibly obesity) could also benefit from DCI treatment. In this sense, DCI acts as a substance, preferably an orally active substance, which stimulates endogenous leptin.

Having now fully described this invention, it will be understood to those of ordinary skill in the art that the same can be performed within a wide and equivalent range of conditions, formulations, and other parameters without affecting the scope of the invention or any embodiment thereof. All patents and publications cited herein are fully incorporated by reference herein in their entirety.

What is claimed is:

1. A method of improving hypothalamic gene expression in a mammal comprising the step of administering to said mammal an effective amount of D-chiro-inositol (DCI), or a derivative or metabolite thereof.

2. A method according to claim 1, wherein said mammal is a human.

3. A method according to claim 1, wherein said derivative or metabolite is selected from the group consisting of D-chiro-inositol phosphates, D-chiro-inositol esters, D-chiro-inositol ethers, D-chiro-inositol acetals, D-chiro-inositol ketals, D-chiro-inositol polysaccharide, D-chiro-inositol phospholipids and metal complexes of D-chiro-inositol.

4. A method according to claim 1, wherein said D-chiro-inositol is administered in combination with an insulin sensitizer.

5. A method according to claim 2, wherein said effective amount is a daily dosage between about 0.07 milligrams and about 3500 milligrams.

6. A method according to claim 5, wherein said D-chiro-inositol is administered orally.

7. A method of controlling the weight of a mammal comprising the step of administering to a mammal in need thereof an effective amount of D-chiro-inositol (DCI), or a derivative or metabolite thereof.

8. A method according to claim 7, wherein said mammal is a human.

9. A method according to claim 7, wherein said derivative or metabolite is selected from the group consisting of D-chiro-inositol phosphates, D-chiro-inositol esters, D-chiro-inositol ethers, D-chiro-inositol acetals, D-chiro-inositol ketals, D-chiro-inositol polysaccharide, D-chiro-inositol phospholipids and metal complexes of D-chiro-inositol.

10. A method according to claim 7, wherein said D-chiro-inositol is administered in combination with an insulin sensitizer.

11. A method according to claim 8, wherein said effective amount is a daily dosage between about 0.07 milligrams and about 3500 milligrams.

12. A method according to claim 11, wherein said D-chiro-inositol is administered orally.

13. A method of increasing serum leptin in a mammal comprising the step of administering to a mammal an effective amount of D-chiro-inositol (DCI), or a derivative or metabolite thereof.

14. A method according to claim 13, wherein said mammal is a human.

15. A method according to claim 13, wherein said derivative or metabolite is selected from the group consisting of D-chiro-inositol phosphates, D-chiro-inositol esters, D-chiro-inositol ethers, D-chiro-inositol acetals, D-chiro-inositol ketals, D-chiro-inositol polysaccharide, D-chiro-inositol phospholipids and metal complexes of D-chiro-inositol.

16. A method according to claim 13, wherein said D-chiro-inositol is administered in combination with an insulin sensitizer.

17. A method according to claim 14, wherein said effective amount is a daily dosage between about 0.07 milligrams and about 3500 milligrams.

18. A method according to claim 17, wherein said D-chiro-inositol is administered orally.

19. A method for the prophylaxis or treatment of diseases or disorders associated with abnormal hypothalamic gene expression comprising the step of administering to a mammal in need thereof an effective amount of D-chiro-inositol (DCI), or a derivative or metabolite thereof.

20. A method according to claim 19, wherein said mammal is a human.

21. A method according to claim 19, wherein said derivative or metabolite is selected from the group consisting of D-chiro-inositol phosphates, D-chiro-inositol esters, D-chiro-inositol ethers, D-chiro-inositol acetals, D-chiro-inositol ketals, D-chiro-inositol polysaccharide, D-chiro-inositol phospholipids and metal complexes of D-chiro-inositol.

22. A method according to claim 19, wherein said D-chiro-inositol is administered in combination with an insulin sensitizer.

23. A method according to claim 20, wherein said effective amount is a daily dosage between about 0.07 milligrams and about 3500 milligrams.

24. A method according to claim 23, wherein said D-chiro-inositol is administered orally.
